(19) United States
(12) Patent Application Publication
(10) Pub. No.: US 2006/0184019 A1
(43) Pub. Date: Aug. 17, 2006

(54) Ultrasonic imaging apparatus

(75) Inventors: Mayumi Ito, Tokyo (JP); Yayoi Abe, Tokyo (JP); Chiori Fujiwara, Tokyo (JP); Tsutomu Yawata, Tokyo (JP)

Correspondence Address: PATRICK W. RASCHE;ARMSTRONG TEASDALE LLP, ONE METROPOLITAN SQUARE, SUITE 2600, ST. LOUIS, MO, 63102-2740, US

(73) Assignee: GE Medical Systems Global Technology Company, LLC

(21) Appl. No.: 11/297,033

(22) Filed: Dec. 8, 2005

(30) Foreign Application Priority Data
Dec. 10, 2004 (JP) 2004-357517

(51) Int. Cl. A61B 8/00; G06K 9/00

(52) U.S. Cl. 600/437; 382/181

(57) ABSTRACT

An ultrasonic imaging apparatus which displays ultrasonic image of a subject, and displays a measurement pattern in the displayed ultrasonic image based on measurement pattern information with respect to a location and a form of the measurement pattern to be input, to perform the image measurement of the ultrasonic image. The ultrasonic imaging apparatus includes a selector device for specifying a location in the ultrasonic image, a measurement pattern storage device for storing morphological information of the measurement pattern when a measurement pattern is present at the specified location, and a measurement pattern reproduction device for reproducing thus stored measurement pattern of the morphological information at a location in the ultrasonic image newly specified using the selector device or an ultrasonic image different from the ultrasonic image.

### BACKGROUND OF THE INVENTION

The present invention is directed to an ultrasonic imaging apparatus, which displays ultrasonic image of a subject, and displays the measurement pattern in said displayed ultrasonic image based on the measurement pattern information with respect to the location and form of the measurement pattern to be input in order to perform the image measurement of said ultrasonic image.

Recently, in the ultrasonic imaging apparatus, the ultrasonic image, as typically represented by the ‘B’ mode image, has finer grains. In addition, not only the morphological examination in the real time basis but also the measurement of the organ anatomy as well as the examination based on the quantitative information derived therefrom can be performed.

In the quantitative examination, the measurement pattern including such as the trace are loaded in the ultrasonic image, and the pattern will be relocated to the target position by using a pointing device such as a track-ball for the image measurement (see for example the patent reference 1).

Furthermore, the quantitative examination by means of the measurement pattern can be performed not only within a single ultrasonic image but also for the data comparison among a plurality of ultrasonic images, each of which may have a different imaging location or the acquisition time. For instance, in the cardiac dynamic examination using an ultrasound imaging apparatus a plurality of ultrasonic images for the atriums and ventricles in the diastolic and systolic times are displayed. For each of these ultrasonic images a measurement pattern will be set to gather and compare the numerical data.

[patent reference 1] JP-A-06-178773 pp 1 & FIG. 1

In accordance with the prior art as have been described above, however, the data comparison using the measurement pattern can be done for the numerical data, and is neither instinctive nor visual. On the other hand, when performing a visual comparison of anatomical morphologies of measurement pattern set to each image by appositing a plurality of ultrasonic images of an imaging location sequencing the dynamism, the morphological comparison between measurement patterns may become too ambiguous to achieve a reliable morphological comparison.

In particular, when measuring the cardiac ultrasonic images using the measurement pattern, the parameters computed from the measurement pattern in a plurality of ultrasonic images can be multitudinous. When comparing among those parameters, it is important for an operator to precisely recognize the comparative morphology of the measurement pattern itself in order to valuably perform an appropriate examination with respect to the values of various parameters.

It is imperative therefore how to achieve an ultrasonic imaging apparatus, which allows the morphological comparison of the measurement pattern set within a plurality of ultrasonic images to be done in a reliable and easier manner.

### SUMMARY OF THE INVENTION

Therefore, an object of the present invention is to provide an ultrasound imaging apparatus, which allows the morphological comparison of the measurement pattern set within a plurality of ultrasonic images to be done in a reliable and easier manner.

To solve the problem described above and to achieve the objects, the ultrasonic imaging apparatus of first aspect in accordance with the invention, which is an ultrasonic imaging apparatus which displays ultrasonic image of a subject, and displays the measurement pattern in said displayed ultrasonic image based on the measurement pattern information with respect to the location and form of the measurement pattern to be input in order to perform the image measurement of said ultrasonic image, is characterized in that it comprises a selector means for specifying the location in said ultrasonic image, a measurement pattern storage means for storing the morphological information of said measurement pattern when a measurement pattern is present at the specified location, and a measurement pattern reproduction means for reproducing thus stored measurement pattern of morphological information at a location in said ultrasonic image newly specified using said selector means or an ultrasonic image different from said ultrasonic image.

In the first aspect of the present invention, the selector means serves to specify a location in an ultrasonic image, and if there is a measurement pattern at the specified location the measurement pattern storage means stores the morphological information of the measurement pattern, and the measurement pattern reproduction means reproduces the measurement pattern at the location in a newly specified ultrasonic image by means of the selector means.

The ultrasonic imaging apparatus of second aspect in accordance with the present invention, is characterized in that said measurement pattern storage means in accordance with first aspect performs said storage in association with said selection.

The ultrasonic imaging apparatus of third aspect in accordance with the present invention, is characterized in that said ultrasonic imaging apparatus in accordance with first or second aspect includes a measurement pattern moving means for translating, rotating, or inverting the measurement pattern at said specified location within said ultrasonic image.

In the third aspect of the present invention, the measurement pattern moving means changes of location of the measurement pattern in the ultrasonic image.

The ultrasonic imaging apparatus in accordance with fourth aspect of the present invention, is characterized in that said selector means in accordance with any one of first to third aspects further includes a cursor to be displayed in said ultrasonic image.

In the fourth aspect of the present invention, the cursor is used for selecting a location.

The ultrasonic imaging apparatus in accordance with fifth aspect of the present invention, said selector means in accordance with the fourth aspect further includes a pointing device for controlling the location of said cursor in said ultrasonic image.

In the fifth aspect of the present invention, the pointing device is equiped for facilitating the cursor position control.

The ultrasonic imaging apparatus in accordance with sixth aspect of the present invention, said pointing device in accordance with the fifth aspect is a track-ball.

In addition, the ultrasonic imaging apparatus in accordance with seventh aspect of the present invention, is characterized in that said measurement pattern reproduction means in accordance with any one of first to sixth aspects, performs said reproduction for a plurality of times.

In the seventh aspect of the present invention, the similar measurement pattern are reproduced for a plurality of times.

The ultrasonic imaging apparatus in accordance with eighth aspect of the present invention, is characterized in that said measurement pattern storage means in accordance with any one of first to seventh aspects includes a sequential storage means for sequentially storing a plurality of said measurement patterns with identification information when there is a plurality of measurement patterns present in said ultrasonic image.

In the eighth aspect of the present invention a plurality of measurement patterns are systematically stored without confusion.

The ultrasonic imaging apparatus in accordance with ninth aspect of the present invention is characterized in that said identification information in accordance with the eighth aspect is numbering information indicative of the sequential number of storing.

The ultrasonic imaging apparatus in accordance with tenth aspect of the present invention, is characterized in that said measurement pattern reproduction means in accordance with any one of eighth or ninth aspect includes a sequential reproduction means for sequentially reproducing said measurement patterns based on said identification information.

In accordance with the tenth aspect of the present invention, a plurality of measurement patterns is systematically reproduced without confusion.

The ultrasonic imaging apparatus in accordance with eleventh aspect of the present invention is characterized in that said measurement pattern storage means in accordance with any one of first to seventh aspects includes a batch storage means for storing at once all of measurement patterns present in said ultrasonic image.

In accordance with the eleventh aspect of the present invention, the batch storage means facilitates to store a plurality of measurement patterns present in the ultrasonic image.

The ultrasonic imaging apparatus in accordance with twelfth aspect of the present invention is characterized in that said measurement pattern reproduction means in accordance with eleventh aspect includes a batch reproduction means for reproducing at once said plurality of measurement patterns.

In accordance with twelfth aspect of the present invention, the batch reproduction means facilitates to reproduce a plurality of measurement patterns in an ultrasonic image.

The ultrasonic imaging apparatus in accordance with thirteenth aspect of the present invention is characterized in that said measurement pattern reproduction means in accordance with any one of first to twelfth aspects includes a magnification rate changer means for changing, when the display magnification rate of said ultrasonic image to be reproduced is different from the display rate of said ultrasonic image used for storing, the size of said measurement pattern to be reproduced in response to the change of said display magnification rate.

In accordance with thirteenth aspect of the present invention, the magnification rate changer means allows the storage and reproduction to be performed across ultrasonic images of different display magnification rate.

In addition, the ultrasonic imaging apparatus in accordance with fourteenth aspect of the present invention, is characterized in that said measurement pattern reproduction means in accordance with any one of first to thirteenth aspects provides an identifiable indication for distinguishing said reproduced measurement pattern from the measurement pattern based on said measurement pattern information to be input.

The ultrasonic imaging apparatus in accordance with fifteenth aspect of the present invention is characterized in that the identifiable indication in accordance with fourteenth aspect is the color of the displayed measurement pattern.

In addition, the ultrasonic imaging apparatus in accordance with sixteenth aspect of the present invention is characterized in that the identifiable indication in accordance with fourteenth aspect is the line width of the displayed measurement pattern.

The ultrasonic imaging apparatus in accordance with seventeenth aspect of the present invention is characterized in that said measurement pattern in accordance with any one of first to sixteenth aspects includes at least one of line segment, circle, ellipse, and the trace of points specified by said selector means.

In accordance with the seventeenth aspect of the present invention, major primary patterns are included in the measurement pattern.

In accordance with the present invention, the selector means specifies the position within an ultrasonic image, and if any measurement pattern is present at the specified location, the measurement pattern storage means will store the morphological info of the measurement pattern, and the measurement pattern reproduction means will reproduce thus stored morphological information of the measurement pattern at the position to be specified later by means of the selector means into an ultrasonic image, so that when performing the image measurement of imaged site where the morphology changes along with time course to compare the morphologies, not only numerical data but also the measurement pattern that indicates the form of imaged site at various temporal phase can be superposed to compare between the morphologies visually and reliably, and ultimately leading to a proper examination of numerical data by the operator.

Further objects and advantages of the present invention will be apparent from the following description of the preferred embodiments of the invention as illustrated in the accompanying drawings.

### BRIEF DESCRIPTION OF THE DRAWINGS

FIG. 1 is a schematic block diagram illustrative of the overview of ultrasonic imaging apparatus.

FIG. 2 is a functional block diagram indicative of the functional structure of an image display function section and controller unit in accordance with the preferred embodiment.

FIG. 3 is a schematic diagram illustrative of an example of measurement pattern.

FIG. 4 is an external appearance view illustrative of a specific example of selector means in accordance with the preferred embodiment.

FIG. 5 is a flow chart indicative of the operation of controller unit in accordance with the preferred embodiment.

FIG. 6 is a schematic diagram illustrative of the storage and reproduction of a measurement pattern in the B mode image.

FIG. 7 is a schematic diagram illustrative of the sequential storage and sequential reproduction of a measurement pattern in the B mode image.

FIG. 8 is a schematic diagram illustrative of the batch storage and batch reproduction of a measurement pattern in the B mode image.

### DETAILED DESCRIPTION OF THE INVENTION

The best mode for carrying out the ultrasonic imaging apparatus in accordance with the present invention will be described in greater details herein below with reference to the accompanying drawings. It should be noted here that the illustrated embodiment are intended not to limit the present invention.

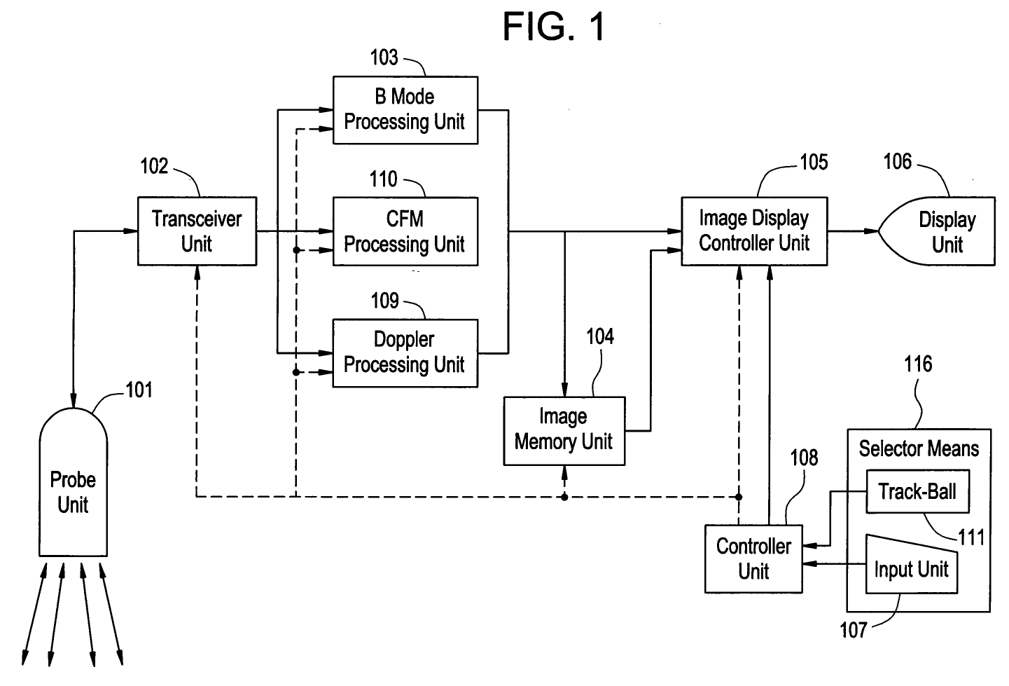

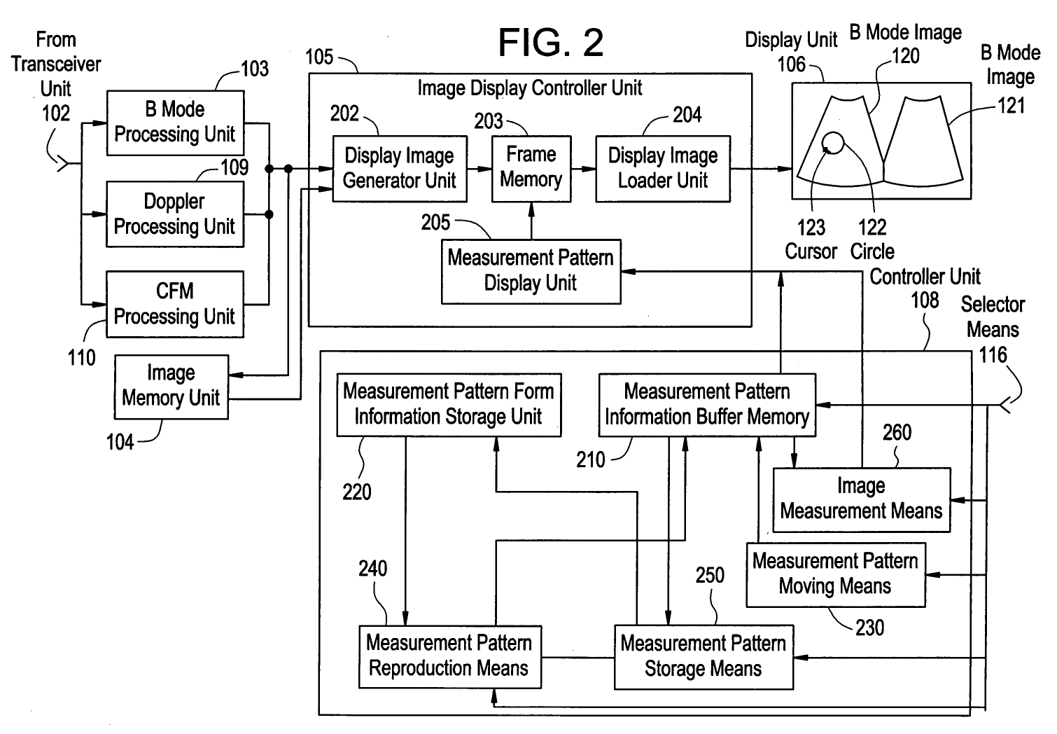

FIG. 1 shows a schematic block diagram indicative of the overview of ultrasonic imaging apparatus in accordance with a preferred embodiment of the present invention. The ultrasonic imaging apparatus has a probe 101, a transceiver unit 102, a B mode processing unit 103, a Doppler processing unit 109, a color flow mapping (CFM) processing unit 110, an image memory unit 104, an image display controller unit 105, a display unit 106, a selector means 116, and a controller unit 108. It is noted here that in FIG. 1 and FIG. 2 which will be described hereinbelow, the signal lines for carrying image information or selection information are indicated by solid lines, while the signal lines for carrying the control information is indicated by dotted lines.

The probe unit 101 is a unit for sending and receiving ultrasonic waves, namely repeatedly transmits the ultrasonic waves to a specific direction in the imaging section of a living body, and receives the ultrasonic signal reflected from the body as the sonic line of time sequence. On the other hand the probe unit 101 sequentially switches the transmission direction of ultrasonic waves to perform the electronic scan. Although not shown in the figure, the probe unit 101 has piezoelectric modules arranged as an array. The probe unit 101 in addition is not limited to be an electronic scanning probe, but may be a probe for mechanical scan.

The transceiver unit 102 is connected to the probe unit 101 with a coaxial cable and generates electric signals for driving the piezoelectric modules in the probe unit 101. The transceiver unit 102 also performs first stage amplification of received ultrasonic signals.

The B mode processing unit 103 generates in real time basis a B mode image from the ultrasonic signals amplified by the transceiver unit 102. More specific processing details may be such as the delayed addition of received ultrasonic signals, Analog-to-Digital conversion, writing digitized information after conversion as the B mode image information to the image memory unit 104 to be described below and so on.

The Doppler processing unit 109 extracts the phase modulation information at the timings corresponding to the sampled area set in the B mode image from within the ultrasonic signals amplified by the transceiver unit 102, to generate as a Doppler image the information on the flow associated with various points in the imaged section in real time basis, such as the velocity, power value, and variance.

The CFM processing unit 110 generates a CFM image, which illustrates the blood flow information included in the ultrasonic signal as red for incoming flow to the probe unit 101 and blue for outgoing flow from the probe unit 101. Note that the term ultrasonic image herein below refers to the image information obtained by transmitting and receiving ultrasonic waves to a subject, including the B mode image described above, CFM image, as well as Doppler image.

The image memory unit 104 is an image memory for accumulating ultrasonic image information such as including B mode image information generated in the B mode processing unit 103, CFM image information generated in the CFM processing unit 110, as well as Doppler image information generated in the Doppler processing unit 109.

The image display controller unit 105 performs control of conversion of displaying frame rate of the ultrasonic image information, mode of image display, and image layout in the display unit 106.

The display unit 106 uses a CRT (cathode ray tube) or LCD (liquid crystal display) and the like to provide visual display of a plurality of image information controlled by the image display controller unit 105 for the operator.

The selector means 116 includes an input unit 107 and a track-ball 111 for inputting control information to the controller unit 108. The input unit 107 may include a keyboard, to communicate the operational input signals from the operator to the controller unit 108. The track-ball 111 is a pointing device for controlling the cursor position on the display screen of the display unit 106. The measurement pattern as will be described below may be specified by the cursor to be displayed in the ultrasonic image on the display unit 106.

The controller unit 108 controls the operation of various units of the ultrasonic imaging apparatus above based on the operational input signal supplied from the input unit 107, and preloaded program and data, to display the ultrasonic image such as B mode image to the display unit 106.

FIG. 2 shows a functional block diagram indicative of a display function section of ultrasonic image and measurement pattern in the controller unit 108 and the image display controller unit 105.

The image display controller unit 105 includes a display image generator unit 202, a frame memory 203, a display image loader unit 204, and a measurement pattern display unit 205. The frame memory 203 is a memory for accumulating display screen information to be displayed on the display unit 106. The display image generator unit 202 generates the image mode to be displayed on the display unit 106 based on the ultrasonic image information from the B mode processing unit 103 and the like. The display image generator unit 202 then writes the image information into the address corresponding to the display position in the frame memory 203. The display image loader unit 204 reads the image information written into the frame memory 203 to display on the display unit 106.

The measurement pattern display unit 205 writes the measurement pattern into the display position in the frame memory 203 based on the measurement pattern information about the location and form of the measurement pattern derived from the controller unit 108. The measurement pattern then will be displayed on the display unit 106. In the display unit 106 shown in FIG. 2, there are two B mode images 120 and 121 shown as examples of ultrasonic images, in addition a cursor 123 and a circle 122 as an example of measurement pattern are shown in the B mode image 120.

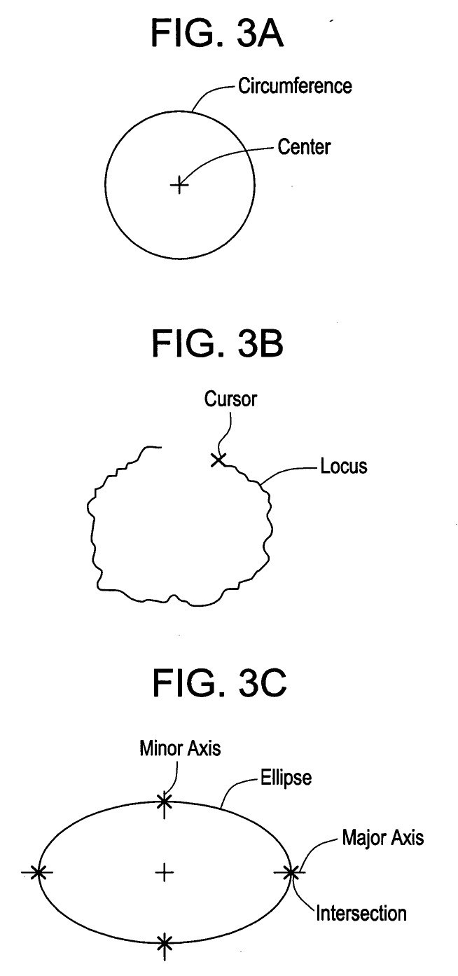

An example of measurement pattern to be shown on the display unit 106 is shown in FIG. 3. FIG. 3 (A) is an example of circle, which is defined by the center position of circle and the position of circumference, both specified by the cursor 123 in the ultrasonic image. FIG. 3 (B) is an example of trace, which displays the trace of the cursor 123 in the ultrasonic image, moved by the track-ball 111 or the like. FIG. 3 (C) is an example of ellipse, the position and the size of which are specified by the cursor 123 in the ultrasonic image. In addition, a caliper indicative of a line segment may be used for a measurement pattern.

The controller unit 108 includes a measurement pattern information buffer memory 210, a measurement pattern form information storage unit 220, a measurement pattern moving means 230, a measurement pattern reproduction means 240, a measurement pattern storage means 250, and an image measurement means 260. The measurement pattern information buffer memory 210 is specified by the cursor 123 at the time when the measurement pattern set mode is selected from the input unit 107. It is a buffer memory for storing the measurement pattern information on the position and form of the measurement pattern. The contents of this buffer memory is update whenever measurement pattern information is input from the selector means 116 and is transferred to the measurement pattern display unit 205.

In the measurement pattern information buffer memory 210, the information of two positional data items of the center position information and the circumference position information of a circle is stored when the circle shown in FIG. 3 (A) is selected; the information of positional data items of many points on the trace is stored when the trace is selected as shown in FIG. 3 (B); and the information of four intersectional positions where the major and minor axes of an ellipse intersect is stored when the ellipse is selected as shown in FIG. 3 (C).

The measurement pattern storage means 250 selects a measurement pattern present at the position of the cursor 123 in the ultrasonic image from the measurement pattern information buffer memory 210 when the measurement pattern edit mode is selected from the input unit 107, and extracts only the morphological information from the measurement pattern information, to store the information in the measurement pattern form information storage unit 220 which is the memory. For instance, when the measurement pattern is a circle, the circle as well as the radius or the diameter of the circle are stored as the form information; information on relative position of the positional information of many points on the trace is stored as the form information when the measurement pattern is a trace; and the information on relative position of the positional information of intersections is stored when the measurement pattern is an ellipse.

The measurement pattern reproduction means 240 is responsive to the selection signal from the input unit 107 to read the form information of the measurement pattern stored in the measurement pattern form information storage unit 220 when the measurement pattern edit mode is selected from the input unit 107, to add the positional information corresponding to the cursor position in the B mode image 120 or 121, and to write it into the measurement pattern information buffer memory 210. Then the measurement pattern display unit 205 generates the measurement pattern in the frame memory 203 to display at the position of the cursor 123 in the display unit 106.

The measurement pattern moving means 230 changes the measurement pattern positional information in the measurement pattern information buffer memory 210, when the measurement pattern edit mode is selected from the input unit 107, in response to the movement of the cursor 123 position, for example with respect to the measurement pattern at the cursor 123 position in the B mode image 120 or 121, to move the measurement pattern so as to follow the position where the cursor 123 is present. The measurement pattern moving means 230 also performs the operation such as rotation, invert of the measurement pattern under the direction from the selector means 116.

The image measurement means 260 performs various measurements by using the position and form information in the measurement pattern information buffer memory 210. For instance, when the circle is selected, it calculates the area of the section encompassed by the circle; when the trace is selected it calculates the area of the section encompassed by the trace; when the ellipse is selected, it calculates the area of the section encompassed by the ellipse. The image measurement means 260 is connected to the measurement pattern display unit 206 of the image display controller unit 105 to transfer the calculation result described above to the measurement pattern display unit 206 to display on the display unit 106.

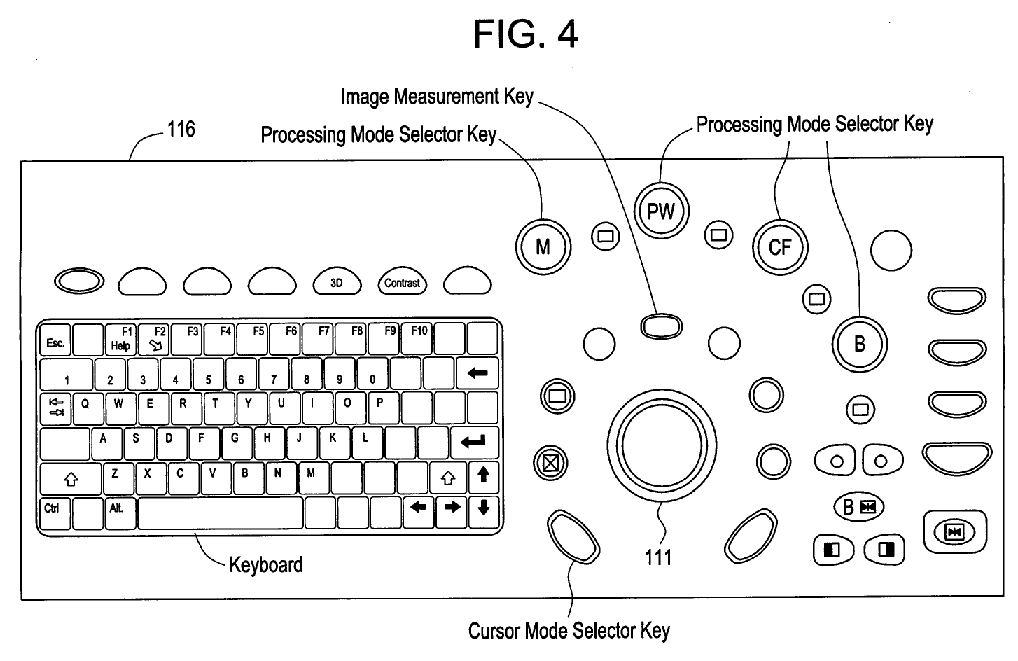

FIG. 4 shows an example of the track-ball 111 and the input unit 107 of the selector means 116. On the left side of the selector means 116 a keyboard is provided and on the right side a track-ball 111 is provided for controlling the position of the cursor 123. Around the track-ball 111 processing mode selection key such as B mode processing and M mode processing in relation to the Doppler processing are placed, and among those keys there are a cursor mode selection key for switching the modes between the measurement pattern set mode and measurement pattern edit mode of the cursor 123, and an image measurement key for invoking the image measurement means 260.

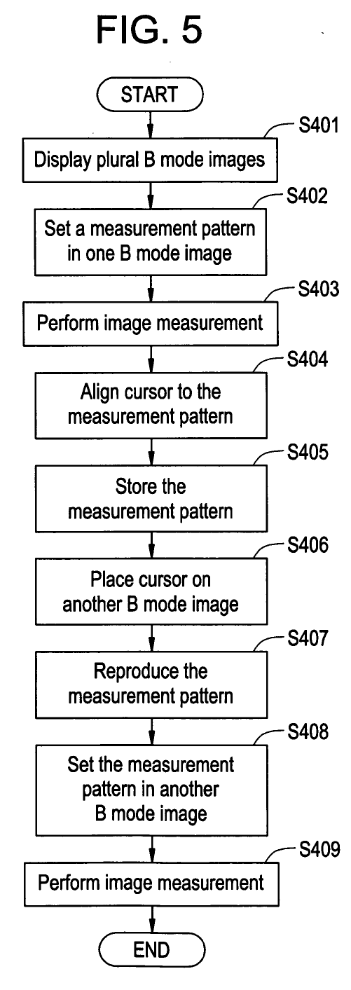

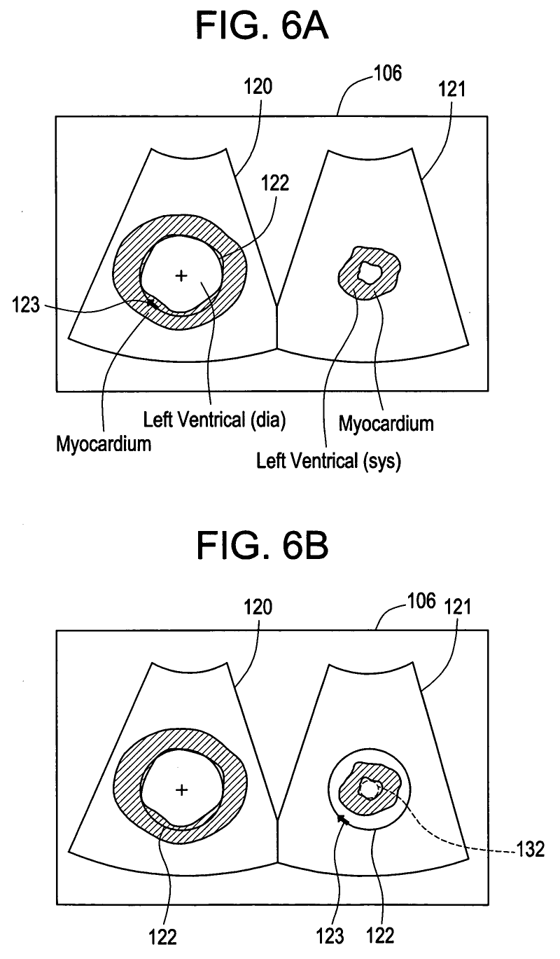

Next, the detailed operation of the measurement pattern storage means 250 and the measurement pattern reproduction means 240 in the controller unit 108 will be described in greater details with reference to FIG. 5. FIG. 5 shows a flow chart illustrating the operation of the measurement pattern storage means 250 and the measurement pattern reproduction means 240 of the controller unit 108. First, the operator instructs to display on the display unit 106 a plurality of still ultrasonic images, for example two B mode images of an imaging site which changes in morphology along with time course (step S401). FIG. 6 shows an example of displaying two B mode images. The B mode images 120 and 121 shown in FIG. 6 illustrates schematically the brachyaxial tomographic plane of the left ventricle of a heart, captured by the ultrasonic imaging apparatus, in which the B mode image 120 of the left hand side shows the left ventricular diastolic phase while the B mode image 121 of the right hand side shows the left ventricular systolic phase. It should be noted here that these two images are selected among many B mode images of cardiac region of a subject that have been imaged in advance using the ultrasonic imaging apparatus and stored in the image memory unit 104.

Then, the operator uses the selector means 116 to select the measurement pattern set mode to set a measurement pattern in the B mode image 120 (step S402). FIG. 6 (A) shows an example of the measurement pattern to be set. Here a circle 122 as measurement pattern is illustrated, which is aligned to the inner wall position of the left ventricle in diastolic phase. The position of the circle 122 may be fitted to the left ventricle by using the measurement pattern moving means 230.

The operator then uses the image measurement means 260 to measure on the image the surface area of inner wall of the left ventricle in diastolic phase based on the circle 122 superimposed thereon (step S403) to obtain some numerical data such as surface area. Although the circle 122 is used as the measurement pattern for the sake of clarity, the trace may be much preferable for the candidate of measurement pattern in order to perform an image measurement of fine precision.

The operator then uses the selector means 116 to select the measurement pattern edit mode to move the cursor 123 on the circle 122 to match with the measurement pattern (step S404), to invoke the measurement pattern storage means 250 by a predetermined keyboard operation such as for example by pressing the CTRL and C keys on the keyboard at the same time, to copy or store the morphological information of the circle 122 to the measurement pattern form information storage unit 220 (step S405).

The operator then moves the cursor 123 to another image, namely the B mode image 121 and place the cursor 123 on the position where the circle 122 is to be reproduced, namely pasted (step S406). The operator thereafter invokes the measurement pattern reproduction means 240 by a predetermined keyboard operation such as for example by pressing the CTRL and V key on the keyboard at the same time, to read out the morphological information of the circle 122 from the measurement pattern form information storage unit 220. Then the measurement pattern reproduction means 240 writes the measurement pattern on the position and form of the circle 122 into the measurement pattern information buffer memory 210 based on the positional information on the cursor 123 from the selector means 116 to paste, namely, reproduce the circle 122 at the position of the cursor 123 in the B mode image 121 (step S407). FIG. 6 (B) shows the circle 122, which is the measurement pattern being reproduced in the B mode image 121. The circle 122 is placed so as to encompass the left ventricle in diastolic phase. The circle 122 may be placed at the optimal location by using the measurement pattern moving means 230 in order to compare with the left ventricle in diastolic phase. A plurality of circles 122 may be reproduced in a B mode image 121 by the similar operation.

The operator thereafter selects the measurement pattern set mode by means of the selector means 116 to set a measurement pattern for example a circle 132 on the left ventricle in the B mode image 121, which indicates the left ventricular systolic phase, while referencing to the circle 122 which is the reproduced measurement pattern (step S408). FIG. 6 (B) shows the circle 132, additionally placed measurement pattern in the B mode image 121, by dotted line. The circle 132 is placed so as to superpose on the inner wall of the left ventricle in systolic phase.

The operator then uses the image measurement means 260 to perform the image measurement such as the area of inner wall of the left ventricle in systolic phase with the circle 132 (step S409) to obtain the numerical data of the left ventricle in systolic phase. The left ventricle in diastolic phase is shown in the B mode image 121 of the systolic phase with the pasted circle 122, so as to allow facilitating the comparison of morphology of the left ventricle in systolic phase to the left ventricle in diastolic phase in an easier and visually reliable manner.

As have been described above, in accordance with the preferred embodiment, since the measurement pattern storage means 250 stores the circle 122 indicative of the size of the inner wall of left ventricle, specified in the B mode image 120 which shows the left ventricular diastolic phase, and the measurement pattern reproduction means 240 reproduces the circle 122 in the B mode image 121 indicative of the left ventricular systolic phase, the comparison of morphology of the inner wall of left ventricle in diastolic and systolic phase can be performed when specifying the circle 132 indicative of the size of the inner wall of left ventricle in systolic phase in the B mode image 121, in an easer and visually reliable manner along with the numerical data from the image measurement means 260 to provide an appropriate determination of the numerical data.

In accordance with the preferred embodiment, although there is only one measurement pattern set in the B mode images 120 and 121, when a plurality of measurement patterns is set in the images, these measurement patterns are specified and stored in the order of sequential number which is served as identification information, to sequentially reproduce in the order of sequential number, so as to perform the storage and reproduction of measurement patterns in an efficient manner.

The measurement pattern storage means 250 in such a case may have a sequential storage means for labeling a sequential number to a plurality of measurement patterns to be stored, while the measurement pattern reproduction means 240 may have a sequential reproduction means for sequentially reading and reproducing thus numbered measurement pattern morphological information.

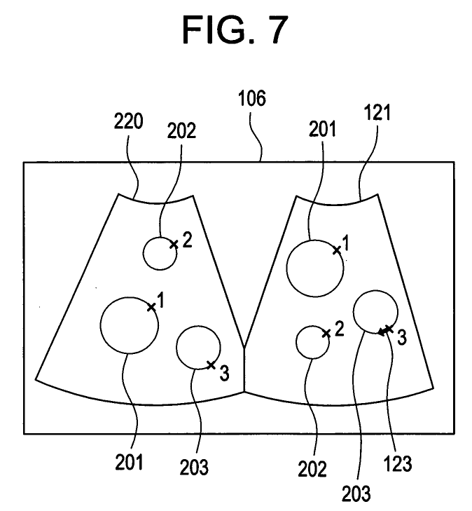

FIG. 7 shows an example of the measurement patterns 211 to 213 being set on the B mode image 220 displayed on the display unit 106. The measurement patterns 211 to 213 are displayed with added sequence number “1” to “3” which are identification information. At the time when selecting one by one the position of the measurement patterns 211 to 213, by means of the cursor 123, the sequential storage means in the measurement pattern storage means 250 stores into the measurement pattern form information storage unit 220 the morphological information of the measurement patterns 211 to 213 along with the number information “1” to “3”. At this point the number information is transferred from the measurement pattern storage means 250 to the measurement pattern reproduction means 240.

The sequential reproduction means in the measurement pattern reproduction means 240 will reproduce the measurement patterns 211 to 213 in the order of number information “1” to “3” for example at the location specified in the B mode image 221 by using the cursor 123. In the B mode image 221 of FIG. 7 thus reproduced measurement patterns 211 to 213 are shown.

In accordance with the preferred embodiment, although there is only one measurement pattern set in the B mode images 120 and 121, there may be cases where a plurality of measurement patterns are set in these images, in which cases the measurement patterns are stored at once and reproduced at once, so as to perform the storage and reproduction in a more effective manner.

The measurement pattern storage means 250 in such a case may have a batch storage means for storing a plurality of measurement patterns at once, and the measurement pattern reproduction means 240 may have a batch reproduction means for reproducing at once a plurality of measurement patterns that have been stored in a batch.

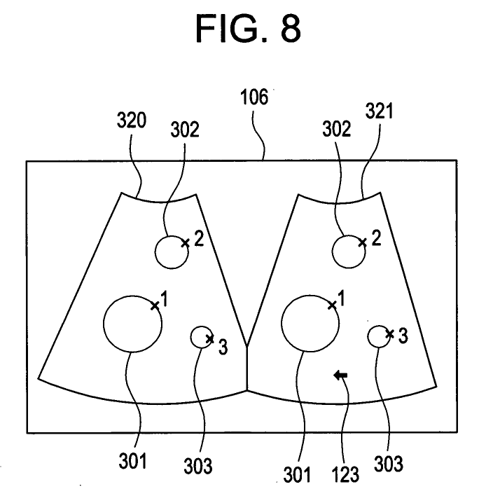

FIG. 8 shows an example of circles 301 to 303 being set as measurement patterns in a B mode image 320 displayed on the display unit 106. In this example by specifying a B mode image 320 by means for example of the cursor 123, the batch storage means of the measurement pattern storage means 250 will store in the measurement pattern form information storage unit 220 the morphological information of the circles 301 to 303 along with the positional information.

Furthermore, the batch reproduction means of the measurement pattern reproduction means 240 may reproduce the circles 301 to 303 at once on a B mode image 321 by specifying the B mode image 321 by means for example of the cursor 123. In the B mode image 321 of FIG. 8 the circles 301 to 303, which have been reproduced at once, are shown.

In addition in accordance with the preferred embodiment, it has been described that the circle 122 set in the B mode image 120 of diastolic phase and the circle 122 reproduced in the B mode image 121 of systolic phase are the same, however the circle 122 reproduced in the B mode image 121 of systolic phase may also have a different color or line width to display as identifiable. By doing this the circle 132 and circle 122 both set in the B mode image 121 of systolic phase can be clearly distinguished each from the other to allow the morphological comparison to be effectuate much easier.

Also in accordance with the preferred embodiment, although the B mode images 120 and 121 has the same display magnification rate of the visualized B mode images, the size of the measurement patterns to be reproduced can be altered in response to the display magnification rate, when the display magnification rate of images are different, to match with the size of B mode images. In this example the measurement pattern reproduction means 240 has a magnification rate changing means, for altering the size of measurement patterns read from the measurement pattern form information storage unit 220 in correspondence with the display magnification rate of the B mode image, such that for example if the B mode image to which a measurement pattern is to be reproduced is displayed magnified to twofold, the measurement pattern to be reproduced in this B mode image will be accordingly magnified to twofold.

Furthermore in accordance with the preferred embodiment although there have been shown examples of left ventricular images in diastolic and systolic phases for the B mode images 120 and 121 that changes in morphology along with time course, images of tumor prior to and after the surgical intervention thereof may be similarly used.

Many widely different embodiments of the invention may be configured without departing from the spirit and the scope of the present invention. It should be understood that the present invention is not limited to the specific embodiments described in the specification, except as defined in the appended claims.

1. An ultrasonic imaging apparatus which displays ultrasonic image of a subject, and displays a measurement pattern in said displayed ultrasonic image based on measurement pattern information with respect to a location and a form of the measurement pattern to be input, to perform the image measurement of said ultrasonic image, said ultrasonic imaging apparatus comprising:
a selector device for specifying a location in said ultrasonic image;
a measurement pattern storage device for storing morphological information of said measurement pattern when a measurement pattern is present at the specified location; and
a measurement pattern reproduction device for reproducing thus stored measurement pattern of the morphological information at a location in said ultrasonic image newly specified using said selector device or an ultrasonic image different from said ultrasonic image.

2. An ultrasonic imaging apparatus according to claim 1, wherein:
said measurement pattern storage device performs said storage in association with said selection.

3. An ultrasonic imaging apparatus according to claim 1, wherein:
said ultrasonic imaging apparatus includes a measurement pattern moving device for translating, rotating, or turning over the measurement pattern at said specified location within said ultrasonic image.

4. An ultrasonic imaging apparatus according to claim 1, wherein:
said selector device further includes a cursor to be displayed in said ultrasonic image.

5. An ultrasonic imaging apparatus according to claim 4, wherein:
said selector device includes a pointing device for controlling a location of said cursor in said ultrasonic image.

6. An ultrasonic imaging apparatus according to claim 5, wherein:
said pointing device is a track-ball.

7. An ultrasonic imaging apparatus according to claim 1, wherein:
said measurement pattern reproduction device performs said reproduction for a plurality of times.

8. An ultrasonic imaging apparatus according to claim 1, wherein:
said measurement pattern storage device includes a sequential storage device for sequentially storing a plurality of said measurement patterns with identification information when there is a plurality of measurement patterns present in said ultrasonic image.

9. An ultrasonic imaging apparatus according to claim 8, wherein:
said identification information is number information indicative of the sequential number of storing.

10. An ultrasonic imaging apparatus according to claim 8, wherein:
said measurement pattern reproduction device includes a sequential reproduction device for sequentially reproducing said measurement patterns based on said identification information.

11. An ultrasonic imaging apparatus according to claim 1, wherein:
said measurement pattern storage device includes a batch storage device for storing at once all of measurement patterns present in said ultrasonic image.

12. An ultrasonic imaging apparatus according to claim 11, wherein:
said measurement pattern reproduction device includes a batch reproduction device for reproducing at once said plurality of measurement patterns.

13. An ultrasonic imaging apparatus according to claim 1, wherein:
said measurement pattern reproduction device includes a magnification rate changer device for changing, when a display magnification rate of said ultrasonic image to be reproduced is different from the display rate of said ultrasonic image used for storing, a size of said measurement pattern to be reproduced in association with the change of said display magnification rate.

14. An ultrasonic imaging apparatus according to claim 1, wherein:
said measurement pattern reproduction device performs identifiable indication for distinguishing said reproduced measurement pattern from the measurement pattern based on said measurement pattern information to be input.

15. An ultrasonic imaging apparatus according to claim 14, wherein:
the identifiable indication is a color of the displayed measurement pattern.

16. An ultrasonic imaging apparatus according to claim 14, wherein:
the identifiable indication is a line width of the displayed measurement pattern.

17. An ultrasonic imaging apparatus according to claim 1, wherein:
said measurement pattern includes at least one of line segment, circle, ellipse, and the trace of points specified by said selector device.
